(19) United States
(12) Patent Application Publication
(10) Pub. No.: US 2023/0117289 A1
(43) Pub. Date: Apr. 20, 2023

(54) COMPOSITIONS COMPRISING METHYLPHENIDATE-PRODRUGS, PROCESSES OF MAKING AND USING THE SAME

(71) Applicant: KEMPHARM, INC., Celebration, FL (US)

(72) Inventors: Sven Guenther, Coralville, IA (US); Guochen Chi, Coralville, IA (US); Travis Mickle, Kissimmee, FL (US)

(21) Appl. No.: 17/798,491

(22) PCT Filed: Feb. 23, 2021
(86) PCT No.: PCT/US2021/019206

§ 371 Date: Aug. 9, 2022

Related U.S. Application Data
(60) Provisional application No. 62/983,614, filed on Feb. 29, 2020.

(51) Int. Cl. C07D 401/12; A61K 9/48

(52) U.S. Cl. CPC C07D 401/12; A61K 9/485; A61K 9/4816; A61K 9/4833; A61K 9/4866

(57) ABSTRACT

The present technology is directed serdexmethylphenidate compounds and methods for synthesizing a compound having the formula

### FEDERALLY SPONSORED RESEARCH OR DEVELOPMENT

[Not Applicable]

### BACKGROUND

Methylphenidate is a psychostimulant which is a chain substituted amphetamine derivative. Similar to amphetamine and cocaine, methylphenidate targets the central nervous system, specifically the dopamine transporter (DAT) and norepinephrine transporter (NET). Methylphenidate is thought to act by increasing the concentrations of dopamine and norepinephrine in the synaptic cleft, as methylphenidate has both dopamine transporter (DAT) and norepinephrine transporter (NET) binding capabilities. Although an amphetamine derivative, the pharmacology of methylphenidate and amphetamine differ, as amphetamine is a dopamine transport substrate whereas methylphenidate works as a dopamine transport blocker. As a norepinephrine and dopamine re-uptake inhibitor, methylphenidate thus blocks re-uptake of dopamine and norepinephrine (noradrenaline) into presynaptic neurons (and possibly stimulates the release of dopamine from dopamine nerve terminals at high doses), thereby increasing the levels of dopamine and norepinephrine in the synapse. In some in vitro studies, methylphenidate has been shown to be more potent as an inhibitor of norepinephrine uptake/re-uptake when compared to dopamine. However, some in vivo studies have indicated that methylphenidate is more potent in potentiating extracellular dopamine concentrations than norepinephrine concentrations. Unlike amphetamine, it has been suggested in the scientific and/or clinical research community that methylphenidate does not seem to significantly facilitate the release of these two monoamine neurotransmitters at therapeutic doses.

Four isomers of methylphenidate are known to exist: d-erythro-methylphenidate, l-erythro-methylphenidate, d-threo-methylphenidate, and 1-threo-methylphenidate. Originally, methylphenidate was marketed as a mixture of two racemates, d/l-erythro-methylphenidate and d/l-threo-methylphenidate. Subsequent research showed that most of the desired pharmacological activity of the mixture is associated with the threo-isomer resulting in the marketing of the isolated threo-methylphenidate racemate. Later, the scientific community determined that the d-threo-isomer is mostly responsible for the stimulant activity. Consequently, new products were developed containing only d-threo-methylphenidate (also known as “d-threo-MPH”).

Stimulants, including methylphenidate (“MPH”), are believed to enhance the activity of the sympathetic nervous system and/or central nervous system (CNS). Stimulants such as MPH and the various forms and derivatives thereof are used for the treatment of a range of conditions and disorders predominantly encompassing, for example, attention deficit hyperactivity disorder (ADHD), attention deficit disorder (ADD), obesity, narcolepsy, appetite suppression, depression, anxiety and/or wakefulness.

Methylphenidate is currently approved by the United States Food and Drug Administration (“FDA”) for the treatment of attention-deficit hyperactivity disorder and narcolepsy. Methylphenidate has also shown efficacy for some off-label indications that include depression, obesity and lethargy. In some aspects, the prodrugs of the present technology may be administered for the treatment of attention-deficit hyperactivity disorder and narcolepsy, or any condition that requires the blocking of the norepinephrine and/or dopamine transporters.

Attention deficit hyperactivity disorder (ADHD) in children has been treated with stimulants for many years. However, more recently, an increase in the number of prescriptions for ADHD therapy in the adult population has, at times, outperformed the growth of the pediatric market. Although there are various drugs currently in use for the treatment of ADHD, including some stimulants and some non-stimulant drugs, methylphenidate (commercially available from, for example, Novartis International AG (located in Basel, Switzerland) under the trademark Ritalin®) is commonly prescribed. Moreover, during classroom trials, non-stimulants have shown to be less effective in improving behavior and attention of ADHD afflicted children than amphetamine derivatives.

Behavioral deterioration (rebound or “crashing”) is observed in a significant portion of children with ADHD as the medication wears off, typically in the afternoon or early evening. Rebound symptoms include, for example, irritability, crankiness, hyperactivity worse than in the un-medicated state, sadness, crying, and in rare cases psychotic episodes. The symptoms may subside quickly or last several hours. Some patients may experience rebound/crashing so severe that treatment must be discontinued. Rebound/crashing effects can also give rise to addictive behavior by enticing patients to administer additional doses of stimulant with the intent to prevent anticipated rebound/crashing negative outcomes and side effects.

Stimulants, such as methylphenidate and amphetamine, have been shown in the conventional art to exhibit noradrenergic and dopaminergic effects that can lead to cardiovascular events comprising, for example, increased heart rate, hypertension, palpitations, tachycardia and in isolated cases cardiomyopathy, stroke, myocardial infarction and/or sudden death. Consequently, currently available stimulants expose patients with pre-existing structural cardiac abnormalities or other severe cardiac indications to even greater health risks and are frequently not used or used with caution in this patient population.

Methylphenidate, like other stimulants and amphetamine derivatives, can become addictive and is prone to substance abuse. Oral abuse has been reported, and euphoria can be achieved through intranasal and intravenous administration.

Dependence on stimulants like cocaine can occur even after usage for a very short period of time due to their potent euphoric effects. For example, early signs of cocaine dependence include difficulty to abstain from cocaine use when it is present or available. Many stimulants including cocaine have a short elimination half-life and thus require frequent dosing to maintain the “high”. Chronic use of supratherapeutic doses of such stimulants may result in numerous mental and/or physical problems. Effects on mood may include anxiety, restlessness, feelings of superiority, euphoria, panic, irritation, and fearfulness. Behavioral symptoms include but are not limited to being extremely talkative, having increased energy, stealing or borrowing money, erratic or odd behavior, violence, lack of participation in activities that were once enjoyable, and reckless and risky behaviors. Examples of physical symptoms of stimulant dependence may include one or more of the following: decreased need to sleep, headaches, nosebleeds, hoarseness, increased heart rate, muscle twitches, malnutrition, increase in body temperature, nasal perforation, abnormal heart rhythms, chronic runny nose, constricting blood vessels, increased heart rate, increased blood pressure, sexual dysfunction, decreased appetite, dilated pupils, risks for contracting Human Immunodeficiency Virus (HIV), hepatitis C and other bloodborne diseases, gangrene of the bowel, cravings, and tremors. Examples of psychological symptoms of stimulant dependence may include one or more of the following: severe paranoia, violent mood swings, break from reality, lack of motivation, psychosis, hallucinations, inability to use sound judgment, and the rationalization of drug use. There is a variety of factors that can trigger or play a role in stimulant use disorder or stimulant dependence. Generally, these factors can be placed into three categories: genetic, biological, and environmental. Research has shown that individuals who have relatives with addiction problems are more likely to develop an addiction including cocaine dependence. The likelihood of becoming stimulant dependent is higher if the relative is a parent. Changes in brain function may be a biological factor that correlates with addiction problems. For example, low dopamine levels in the brain may result in an individual to abuse substances with the goal to attain pleasurable feelings. Environmental factors include but are not limited to unpredictable situations in the home lives of an individual; stressors, such as child abuse, the loss of a loved one, or other traumatic events.There is a need in the art for forms of methylphenidate that have a slow gradual increase in methylphenidate blood/brain concentrations until peak concentrations are achieved, or a slow gradual decrease of methylphenidate blood/brain concentrations after peak concentrations, or both. Not wishing to be bound by any particular theory, it is possible that slow onset of stimulant concentrations can decrease cardiovascular side effects, and slow elimination can decrease rebound effects. It has also been suggested that a larger increase in synaptic dopamine per time unit (i.e., higher rate of dopamine increase) results in more robust and intense euphoric effect. A slow increase in methylphenidate brain concentration produces a low rate of increase in synaptic dopamine and thus, may result in less rewarding and reinforcing effects. Without wishing to be bound by any particular theory, it has also been suggested that high occupancy of dopamine transporter receptors may decrease the rewarding and reinforcing effects of additional doses of stimulants like cocaine. This could be accomplished, for example, by repeated administration of large doses of a form of methylphenidate with a slow onset that does not result in euphoria.

There is also a need in the art for forms of methylphenidate that can provide a more rapid onset of methylphenidate blood/brain concentrations. Not wishing to be bound by any theory, certain indications may require a large and fast initial spike in blood and/or brain concentration of methylphenidate to provide to the subject sufficient efficacy, while other indications may require lower blood/brain concentrations of methylphenidate, but a small therapeutic amount of a form of methylphenidate with rapid onset may still be beneficial to provide fast efficacy when needed.

There is a further need in the art for forms of methylphenidate that can provide flexibility in dosing regimens. For example, a single daily dose form of methylphenidate in a composition that can provide both immediate and extended release PK profiles would be highly desirable.

There is an additional need in the art for forms of methylphenidate that can maintain the pharmacological benefit when administered, in particular via the oral route, but which preferably have no or a substantially decreased pharmacological activity when administered through injection or intranasal routes of administration.

### BRIEF SUMMARY

The present technology provides a particular d-threo-methylphenidate (“d-MPH”, “d-methylphenidate”, “dexmethylphenidate”) conjugate, or pharmaceutically acceptable salts thereof, to provide, for example, at least one single daily dose form of a d-methylphenidate conjugate in a composition with unconjugated methylphenidate that can provide both immediate and extended release PK profiles when compared to unconjugated d-methylphenidate. The release profile in some instances provides the ability of the prodrug or composition to be administered using dosing regimens that are not easily utilized with the unconjugated d-methylphenidate. In some aspects, the unconjugated methylphenidate in the composition can be d-methylphenidate, 1-methylphenidate, or a mixture thereof, and/or a therapeutic or pharmaceutically acceptable salt thereof.

In another aspect, the present technology provides a prodrug composition comprising at least one conjugate of d-methylphenidate having a structure of Formula I:

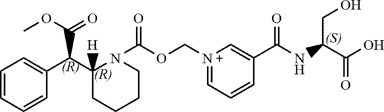

and unconjugated methylphenidate, wherein the unconjugated methylphenidate comprises d-methylphenidate.

In another aspect, the present technology provides at least one prodrug composition comprising at least one conjugate, wherein the at least one conjugate is d-methylphenidate-CO₂CH₂-nicotinoyl-L-Ser (Formula I), or pharmaceutically acceptable salts thereof, and unconjugated methylphenidate.

In a further aspect, the present technology provides a composition comprising unconjugated methylphenidate and at least one conjugate, wherein the at least one conjugate has at least two or more chiral centers and the composition is optically active.

In yet another aspect, the present technology provides a method for chemically synthesizing a d-methylphenidate-CO₂CH₂-nicotinoyl-L-Ser conjugate of the present technology by performing the appropriate steps to conjugate d-methylphenidate to the -CO₂CH₂-nicotinoyl-L-Ser ligand.

In further aspects, some aspects of the compositions of the present technology, comprising (a) the conjugate of Formula I and/or its pharmaceutically acceptable salt(s) and (b) unconjugated methylphenidate (comprising d-methylphenidate) and/or its pharmaceutically acceptable salts, unexpectedly exhibit increased plasma concentrations of d-methylphenidate after Tₘₐₓ (or later) resulting in a controlled or extended-release profile as compared to an equimolar dose of unmodified d-methylphenidate.

In another aspect, some aspects of the compositions of the present technology, comprising (a) the conjugate of Formula I and/or its pharmaceutically acceptable salt(s) and (b) unconjugated methylphenidate (comprising d-methylphenidate) and/or its pharmaceutically acceptable salts, exhibit increased plasma concentrations of d-methylphenidate from about 0 to about 4 hours following oral administration as compared to an orally administered equimolar dose of unconjugated d-methylphenidate released from Concerta®.

In a further aspect, some aspects of the compositions of the present technology, comprising (a) the conjugate of Formula I and/or its pharmaceutically acceptable salt(s) and (b) unconjugated methylphenidate (comprising d-methylphenidate) and/or its pharmaceutically acceptable salts, exhibit increased plasma concentrations of d-methylphenidate for up to about 4 hours following oral administration as compared to an orally administered equimolar dose of unconjugated d-methylphenidate released from Concerta®.

In yet a further aspect, some aspects of the compositions of the present technology, comprising (a) the conjugate of Formula I and/or its pharmaceutically acceptable salt(s) and (b) unconjugated methylphenidate and/or its pharmaceutically acceptable salts, surprisingly exhibit less interpatient variability in the oral pharmacokinetic (PK) profile when compared to unconjugated d-methylphenidate.

In yet another aspect, some aspects of the compositions of the present technology are provided in an amount sufficient to provide an increased AUC when compared to unconjugated d-methylphenidate when administered orally at equimolar doses.

In still further aspects, some aspects of the compositions of the present technology are provided in an amount sufficient to provide a surprisingly lower Cₘₐₓ and a lower AUC but significantly increased partial AUCs for time periods after Tₘₐₓ (or later) of the released d-methylphenidate as compared to unconjugated d-methylphenidate when administered orally at equimolar doses.

In yet further aspects, some aspects of the compositions of the present technology are provided in an amount sufficient to provide a lower Cₘₐₓ and a similar AUC, but significantly increased partial AUCs for time periods after Tₘₐₓ (or later) of the released d-methylphenidate as compared to unconjugated d-methylphenidate when administered orally at equimolar doses.

In yet an alternative aspect, some aspects of the compositions of the present technology are believed to provide reduced side effects as compared to unconjugated d-methylphenidate when administered at equimolar doses, and are also contemplated in some alternative aspects to provide reduced abuse potential as compared to unconjugated d-methylphenidate.

In addition, some aspects of the compositions of the present technology are also believed to unexpectedly provide an amount sufficient to provide an extended Tₘₐₓ when compared to unconjugated d-methylphenidate when administered at equimolar doses, and/or provide an equivalent Tₘₐₓ when compared to unconjugated d-methylphenidate when administered orally at equimolar doses.

Further, some aspects of the compositions of the present technology are also believed to unexpectedly provide an amount sufficient to provide a shorter Tₘₐₓ when compared to an orally administered equimolar dose of unconjugated d-methylphenidate released from Concerta®.

In addition, some aspects of the compositions of the present technology are also believed to unexpectedly provide an amount sufficient to provide a longer half-life (T_{½}) when compared to an orally administered equimolar dose of unconjugated d-methylphenidate released from Concerta®.

In addition, some aspects of the compositions of the present technology are also believed to unexpectedly provide an amount sufficient to provide a longer T_{½} compared to unconjugated d-methylphenidate when administered orally at equimolar doses.

Moreover, the present technology provides at least one method of treating one or more subjects (human or animal) or patients (human or animal) having at least one disease, disorder or condition mediated by controlling, preventing, limiting, or inhibiting neurotransmitter uptake/re-uptake or hormone uptake/re-uptake comprising orally administering to one or more subjects or patients a pharmaceutically and/or therapeutically effective amount of a composition of the present technology, comprising unconjugated methylphenidate and/or its pharmaceutically acceptable salts, and a conjugate of Formula I and/or its pharmaceutically acceptable salts.

In still yet a further aspect, the present technology provides at least one method of treating a subject (human or animal) having at least one disorder or condition requiring stimulation of the central nervous system of the subject, comprising orally administering a pharmaceutically effective amount of a composition of the present technology, comprising unconjugated methylphenidate and/or its pharmaceutically acceptable salts and a conjugate of Formula I and/or its pharmaceutically acceptable salts, wherein the administration treats at least one disorder or condition requiring stimulation of the central nervous system of the subject.

In still yet a further aspect, the present technology provides at least one method of treating a subject (human or animal) having at least one disorder or condition requiring stimulation of the central nervous system of the subject, comprising orally administering a therapeutically effective amount of a composition of the present technology, comprising unconjugated methylphenidate and/or its pharmaceutically acceptable salts and a conjugate of Formula I and/or its pharmaceutically acceptable salts, wherein the administration treats at least one disorder or condition requiring stimulation of the central nervous system of the subject.

In yet another aspect, the present technology provides one or more methods of administering to a subject a composition comprising at least one conjugate of d-methylphenidate and unconjugated methylphenidate, wherein the administration decreases the number of and/or the amount of metabolites produced when compared with unconjugated d-methylphenidate. In other aspects, the one or more methods of administering the composition of the present technology is believed to decrease the exposure of the subject to ritalinic acid when compared with unconjugated d-methylphenidate. It is desirable to minimize exposure to metabolites, such as ritalinic acid, that do not contribute significantly to the intended therapeutic effect because of potential side effects or toxicity that may still occur as a result of potential secondary pharmacological effects of the metabolite. In some aspects, compositions of the present technology may reduce overall exposure to ritalinic acid by about 25% to about 75%.

In yet a further aspect, the compositions of the present technology are believed to provide an increased water solubility of the d-methylphenidate-based conjugate or prodrug compared to unconjugated d-methylphenidate. In another aspect, the increased water solubility is believed to allow for the compositions to be formed into certain dosage forms at higher concentrations, dosage strengths, or higher dose loading capacities than unconjugated d-methylphenidate. In some aspects, such dosage forms include, for example, oral thin films or strips.

In still yet a further aspect, the administration to a patient (human or animal) of the d-methylphenidate-based compositions comprising d-methylphenidate conjugates and unconjugated methylphenidate are believed to provide a reduced interpatient variability of d-methylphenidate plasma concentrations, and are believed to have an improved safety profile when compared to unconjugated d-methylphenidate.

In yet another alternative aspect, the present technology provides at least one method of treating attention-deficit hyperactivity disorder comprising administering to a subject or patient a pharmaceutically and/or therapeutically effective amount of a composition comprising at least one d-methylphenidate conjugate and unconjugated methylphenidate, wherein the administration treats attention-deficit hyperactivity disorder in the subject.

In yet another alternative aspect, the present technology provides at least one method of treating eating disorder, binge eating disorder, obesity, narcolepsy, chronic fatigue, sleep disorder, excessive daytime sleepiness (EDS), cocaine dependence, or stimulant dependence in a subject or patient comprising administering to a subject or patient a pharmaceutically and/or therapeutically effective amount of a composition comprising at least one d-methylphenidate conjugate and unconjugated methylphenidate, wherein the administration treats an eating disorder, binge eating disorder, obesity, narcolepsy, chronic fatigue, sleep disorder, excessive daytime sleepiness (EDS), cocaine dependence, or stimulant dependence in a subject or patient.

In another further aspect, the present technology provides a composition for treating at least one subject or patient having a disorder or condition requiring stimulation of the central nervous system of the subject, wherein the composition comprises unconjugated methylphenidate and a d-methylphenidate conjugate, and wherein the composition has a reduced abuse potential when administered compared to unconjugated d-methylphenidate.

In a further aspect, the compositions of the present technology are contemplated to exhibit reduced or prevented pharmacological activity when administered by parenteral routes, or reduced plasma or blood concentration of released d-methylphenidate when administered intranasally, intravenously, intramuscularly, subcutaneously or rectally as compared to free unconjugated d-methylphenidate when administered at equimolar amounts.

In some aspects, the compositions of the present technology have an extended or controlled release profile as measured by plasma concentrations of released d-methylphenidate when compared to unconjugated d-methylphenidate when administered orally at equimolar doses. In some aspects, the plasma concentration of d-methylphenidate released from the conjugate of the composition would increase more slowly and over a longer period of time after oral administration, resulting in a delay in peak plasma concentration of released d-methylphenidate and in a longer duration of action when compared to unconjugated d-methylphenidate. In further aspects, the controlled release profile of d-methylphenidate of the composition would have a Tₘₐₓ that is about equal to unconjugated d-methylphenidate but provides plasma concentrations of d-methylphenidate that are sustained for a longer period of time as compared to unconjugated d-methylphenidate.

In other aspects, the composition has a lower AUC and lower Cₘₐₓ, but an equivalent Tₘₐₓ and higher d-methylphenidate plasma concentrations in the second half of the day when administered orally once per day compared to unconjugated d-methylphenidate administered orally once per day.

In another aspect, the present technology provides a pharmaceutical kit comprising a specified amount of individual doses in a package, each dose comprising a pharmaceutically and/or therapeutically effective amount of a composition comprising at least one conjugate of d-methylphenidate and unconjugated methylphenidate. The pharmaceutical kit also comprises instructions for use.

In another further aspect, the present technology provides an oral formulation. The oral formulation may comprise a therapeutic dose of (a) d-threo-methylphenidate (S)-serine conjugate and/or its pharmaceutically acceptable salts, and (b) unconjugated methylphenidate and/or its pharmaceutically acceptable salts.

In certain aspects, compositions of the present technology comprising unconjugated methylphenidate and at least one conjugate of d-methylphenidate can be used in neonatal, pediatric, adolescent, adult and/or geriatric subjects with ADHD. For example, in some aspects, the present compositions can be used for a once-daily dosing with a potentially improved onset and a long duration of action, attributes that may benefit neonatal, pediatric and/or adolescent subjects with ADHD.

### BRIEF DESCRIPTION OF THE DRAWINGS

Aspects of the present disclosure will now be described, by way of example only, with reference to the attached Figures, wherein:

FIG. 1 illustrates a flow diagram of (S)-tert-butyl 3-(tert-butoxy)-2-(nicotinamido)-propanoate synthesis 100, according to some aspects. Nicotinic acid is reacted with L-Ser(‘Bu)O’Bu HCl (O-tert-butyl-L-serine tert-butyl ester hydrochloride) in the presence of triethylamine in MTBE and acetonitrile, according to one aspect.

FIG. 2 illustrates a flow diagram of 1^{st} serdexmethylphenidate chloride intermediate (1^{st} SDX intermediate) synthesis, according to one aspect.

FIG. 3 illustrates a flow diagram of 2^{nd} serdexmethylphenidate chloride intermediate (2^{nd} SDX intermediate) synthesis, according to one aspect..

FIG. 4 illustrates a flow diagram of crude serdexmethylphenidate chloride synthesis, according to one aspect..

FIG. 5 illustrates a flow diagram of a first recrystallization for purification and isolation of an SDX drug substance, according to one aspect.

FIG. 6 illustrates a flow diagram of a second recrystallization for purification and isolation of an SDX drug substance, according to one aspect.

FIG. 7 illustrates re-slurry of crystallized SDX solids, according to one aspect.

FIG. 8 illustrates manufacture method of SDX/d-MPH capsules, according to one aspect.

### DETAILED DESCRIPTION OF THE INVENTION

Various aspects will be described in detail with reference to the drawings, wherein like reference numerals represent like parts and assemblies throughout the several views. It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a,” “an,” and “the” include plural reference unless the context clearly indicates otherwise.

Unless defined otherwise, all technical and scientific terms used herein have the same meaning as commonly understood to one of ordinary skill in the art to which this disclosure belongs.

The present technology provides one or more compositions comprising serdexmethylphenidate chloride (SDX). The composition has beneficial properties as further described herein.

The use of the term “methylphenidate” herein is meant to include any of the stereoisomer forms of methylphenidate, including the four stereoisomers: d-erythro-methylphenidate, l-erythro-methylphenidate, d-threo-methylphenidate and l-threo-methylphenidate and the salts and derivatives thereof. Methylphenidate is interchangeable with methyl phenyl(piperidin-2-yl)acetate. The term “methylphenidate” includes all salt forms. Methylphenidate is also known by its trade name Concerta® (commercially available from Janssen Pharmaceuticals, Inc., Beerse, Belgium), Ritalin®, Ritalin® SR, Methylin®, Methylin® ER (all commercially available from Novartis International AG, of Basil, Switzerland). The methylphenidate used in the present technology can be any stereoisomer of methylphenidate, including, but not limited to, d-erythro-methylphenidate, l-erythro-methylphenidate, d-threo-methylphenidate and l-threo-methylphenidate. In a preferred aspect, the conjugates contain a single d-threo-methylphenidate isomer. In another aspect, the prodrug conjugates are optically active single isomers thereof.

The use of the term “unconjugated methylphenidate” means methyl 2-phenyl-2-(piperidin-2-yl)acetate and salts thereof.

Stereoisomers, used hereinafter, means that two molecules are described as stereoisomers of each other if they are made of the same atoms, connected in the same sequence, but the atoms are positioned differently in space. The difference between two stereoisomers can only be seen when the three-dimensional arrangement of the molecules is considered.

Bioavailability, used hereinafter, means the proportion of a drug or other substance that enters the circulation over time when introduced into the body and so is able to have an active effect.

Cₘₐₓ, used hereinafter, is a term used in pharmacokinetics and refers to the maximum (or peak) plasma concentration that a drug achieves in a specified compartment or test area of the body after the drug has been administered and before the administration of a second dose.

Tₘₐₓ, used hereinafter, is the term used in pharmacokinetics to describe the time at which the Cₘₐₓ is observed. After an intravenous administration, Cₘₐₓ and Tₘₐₓ are closely dependent on the experimental protocol, since the concentrations are always decreasing after the dose.

As known to those skilled in the art, the term “Steady State” means the state in which the overall intake of a drug is in approximate dynamic equilibrium with its elimination. At steady state, total drug exposure does not change significantly between successive dosing periods. Steady state is typically achieved following a time period about 4-5 times the half-life of a drug after regular dosing was started.

The use of the term “dose” means the total amount of a drug or active component taken each time by an individual subject.

As used herein, the term “subject” means a human or animal, including but not limited to a human or animal patient.

The term “patient” means a human or animal subject in need of treatment.

The use of the term “interpatient variability” means an estimate of the levels of pharmacokinetic variability between different individuals receiving the same dose of the same drug. The estimate can be made, for example, by calculating the coefficient of variation (CV) of certain pharmacokinetics parameters including, for example, Cₘₐₓ, AUCₗₐₛₜ, AUC_{inf}, and Tₘₐₓ. When comparing interpatient variability between different drugs or between the same drug(s) in different formulations, lower CV indicates reduced interpatient variability and higher CV indicates increased interpatient variability.

“Coefficient of variant” (CV) is a term used in statistics and is calculated based on the following formula: CV = standard deviation/mean* 100.

AUCₗₐₛₜ is a term used in pharmacokinetics to describe the area under the curve in a plot of drug concentration in blood, serum, or plasma vs time from time=0 (or or predose) to the time of the last measurable drug concentration.

AUC_{inf} is a term used in pharmacokinetics to describe the area under the curve in a plot of drug concentration in blood, serum, or plasma vs time from time=0 (or or predose) to infinity.

Molar equivalent as used hereinafter, means an equal number of moles of the substance as the number of moles in a certain mass (weight) or volume, e.g. a dose of d-methylphenidate that is molar equivalent to a dose of about 0.1 mg d-methylphenidate hydrochloride per day would provide the same number of moles of d-methylphenidate as from 0.1 mg of d-methylphenidate hydrochloride.

As used herein, the phrases such as “decreased,” “reduced,” “diminished” or “lowered” are meant to include at least about a 10% change in pharmacological activity, area under the curve (AUC) and/or peak plasma concentration (Cₘₐₓ) with greater percentage changes being preferred for reduction in abuse potential and overdose potential of the conjugates of the present technology as compared to unconjugated methylphenidate. For instance, the change may also be greater than about 10%, about 15%, about 20%, about 25%, about 35%, about 45%, about 55%, about 65%, about 75%, about 85%, about 95%, about 96%, about 97%, about 98%, about 99%, or increments therein.

“Pharmaceutically effective amount” as used herein means an amount that has a pharmacological effect. A “pharmaceutically acceptable salt” as used herein is a salt of the d-methylphenidate conjugate or unconjugated methylphenidate or both which, when used in a pharmaceutically effective amount, has at least one pharmacological effect.

“Therapeutically effective amount” as used herein means an amount effective for treating a disease or condition. A “therapeutically acceptable salt” as used herein is a pharmaceutically acceptable salt of the d-methylphenidate conjugate or unconjugated methylphenidate or both in the composition of the present technology, which, when used in a therapeutically effective amount, is effective for treating a disease, condition, or syndrome.

As used herein, the term “attention deficit hyperactivity disorder” (ADHD) encompasses various sub-types of ADHD including, for example, subjects who do not show or only show weak symptoms of hyperactivity or impulsiveness, or for example, subjects who are predominately inattentive (formerly attention deficit disorder (ADD)).

As used herein, the term “prodrug” refers to a substance that is inactive or has reduced pharmacological activity but is converted to an active drug by a chemical or biological reaction in the body. In the present technology, the prodrug is a conjugate of at least one drug, d-methylphenidate, a linker, and a nicotinoyl-L-serine moiety. Thus, the conjugates of the present technology are prodrugs and the prodrugs of the present technology are conjugates.

Prodrugs are often useful because, in some aspects, they may be easier to administer or process than the parent drug. They may, for instance, be more bioavailable by oral administration whereas the parent drug is not. The prodrug may also have improved solubility in water and/or other solvents over the parent drug. An aspect of a prodrug would be a d-methylphenidate conjugate that is metabolized to the active moiety. In certain aspects, upon in vivo administration, a prodrug is chemically converted to the biologically, pharmaceutically or therapeutically more active form of the compound. In certain aspects, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug is designed to alter the metabolism or the transport characteristics of a drug—the changes typically varying with route of administration—in certain aspects, to mask side-effects or toxicity, to improve bioavailability and/or water solubility, to improve the flavor of a drug or to alter other characteristics or properties of a drug in other discrete aspects.

The d-methylphenidate prodrug can be prepared so as to have a variety of different chemical forms including chemical derivatives or salts. Such d-methylphenidate prodrugs can also be prepared to have different physical forms. For example, the d-methylphenidate prodrug may be amorphous, may have different crystalline polymorphs, or may exist in different solvation or hydration states, such as semi-hydrates, monohydrates, hydrates (nH₂O, when n is 0.5, 1, 2..). Such polymorphs can be produced by, e.g., using crystallization conditions to isolate a free-base and salt forms and/or by ball-milling such forms.

By varying the form of the d-methylphenidate prodrug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility. Accordingly, variation of the crystalline state of the d-methylphenidate prodrug is one of many ways in which to modulate the physical properties thereof.

A co-crystal is a multiple component crystal containing two or more non-identical molecules in which all components are solid under ambient conditions (i.e., 22° C., 1 atmosphere of pressure) when in their pure form. The components comprise a target molecule (i.e., a d-methylphenidate prodrug) and a molecular co-crystal former that coexist in the co-crystal at the molecular level within a single crystal.

Co-crystals that comprise two or more molecules (co-crystal formers) Jmarsson et al., 2004) that are solids under ambient conditions represent a long-known class of compounds (see Wohler, 1844). However, co-crystals remain relatively unexplored. A Cambridge Structural Database (CSD) (Allen et al., 1993) survey reveals that co-crystals represent less than 0.5% of published crystal structures. Nevertheless, their potential impact upon pharmaceutical (e.g., nutraceutical) formulation (Vishweshwar et al., 2006; Li et al., 2006; Remenar et al., 2003; and Childs et al., 2004) and green chemistry (Anastas et al., 1998) is of topical and growing interest. In particular, the fact that all co-crystal components are solids under ambient conditions has important practical considerations because synthesis of co-crystals can be achieved via solid-state techniques (mechanochemistry) (Shan et al., 2002), and chemists can execute a degree of control over the composition of a co-crystal since they can invoke molecular recognition, especially hydrogen bonding, during the selection of co-crystal formation. Those features distinguish co-crystals from solvates which are another broad and well-known group of multiple component compounds. Solvates are much more widely characterized than co-crystals (e.g., 1652 co-crystals are reported in the CSD versus 10,575 solvates; version 5.27 (May 2006) 3D coordinates, RO.075, no ions, organics only).

It would be advantageous to have new forms of d-methylphenidate prodrugs that have improved properties. Specifically, it is desirable to identify improved forms of d-methylphenidate prodrugs that exhibit significantly improved properties including increased aqueous and/or solvent solubility and stability. Further, it is desirable to improve the processability, or preparation of pharmaceutical formulations. For example, needle-like crystal forms or habits of d-methylphenidate prodrug can cause aggregation, even in compositions where the d-methylphenidate prodrug is mixed with other substances, such that a non-uniform mixture is obtained. It is also desirable to increase or decrease the solution rate of d-methylphenidate prodrug-containing pharmaceutical compositions in water or other solvents, increase or decrease the bioavailability of orally-administered compositions, and provide a more rapid or more delayed onset to therapeutic effect. It is also desirable to have a form of the d-methylphenidate prodrug which, when administered to a subject, reaches a peak plasma level faster or slower, has a longer lasting therapeutic plasma concentration, and higher or lower overall exposure when compared to equivalent amounts of the d-methylphenidate prodrug in its presently-known form. The improved properties discussed above can be altered in a way which is most beneficial to a specific d-methylphenidate prodrug for a specific therapeutic effect.

The d-methylphenidate prodrug or conjugate of the present technology and the unconjugated methylphenidate can be either a positively charged (cationic) molecule, or a pharmaceutically acceptable anionic or cationic salt form or salt mixtures with any ratio between positive and negative components. These anionic salt forms can include, but are not limited to, for example, acetate, /-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, /-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. In the preferred aspects, the anionic salt form is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite.

In some aspects, the salt form of the conjugate is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, and nitrite. In some aspects, the salt form of the unconjugated methylphenidate is selected from the group consisting hydrochloride, hydrobromide, hydroiodide, formate, mesylate, tartrate, salicylate, sulfate, citrate, nitrate, hydrogen sulfite, propionate, benzene sulfonate, and acetate.

The cationic salt forms can include, but are not limited to, for example, sodium, potassium, calcium, magnesium, lithium, cholinate, lysinium, or ammonium.

Without wishing to be limited to the following theory, it is believed that the prodrugs/conjugates of the present technology undergo rate determining enzyme hydrolysis in vivo, which subsequently leads to a cascade reaction resulting in rapid formation of d-methylphenidate and the respective ligands, metabolites thereof and/or derivatives thereof. The prodrug conjugates of the present technology are non-toxic or have very low toxicity at the given dose levels and are preferably known drugs, natural products, metabolites, or GRAS (Generally Recognized As Safe) compounds (e.g., preservatives, dyes, flavors, etc.) or non-toxic mimetics or derivatives thereof.

### Synthetic Schemes for Making Serdexmethylphenidate Chloride

Abbreviations for the components of the compositions of the present technology include: SDX stands for serdexmethylphenidate chloride; MPH stands for methylphenidate; d-MPH stands for methylphenidate hydrochloride; CMCF for chloromethyl chloroformate; MTBE stands for methyl-t-butyl ether; MIBK stands for 4-methyl-2-pentanone ^{t}Bu stands for tert-butyl; Ph stands for phenyl; T₃P stands for propylphosphonic anhydride; ACN stands for acetonitrile.

In some aspects, the serdexmethylphenidate conjugate is the ionic salt serdexmethylphenidate chloride represented by Formula I:

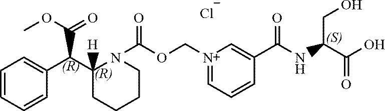

In preferred aspects of the compositions of the present technology, the d-methylphenidate active is derived from two sources, serdexmethylphenidate chloride, and unconjugated methylphenidate and/or its pharmaceutically acceptable salts.

In some aspects, serdexmethylphenidate chloride is synthesized in four stages starting from dexmethylphenidate hydrochloride (d-MPH), chloromethyl chloroformate (CMCF), and (S)-tert-butyl 3-(tert-butoxy)-2-(nicotinamido)-propanoate, shown below:

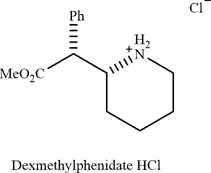

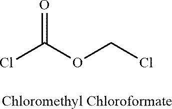

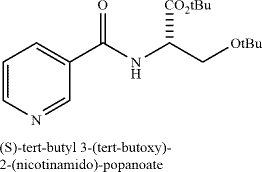

### Preparation of (S)-tert-butyl 3-(tert-butoxy)-2-(nicotinamido)-propanoate

In some aspects, (S)-tert-butyl 3-(tert-butoxy)-2-(nicotinamido)-propanoate is prepared according to Scheme 1.

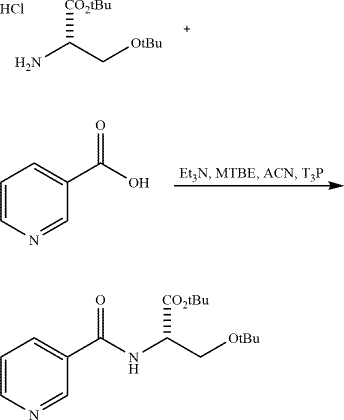

(S)-tert-butyl 3-(tert-butoxy)-2-(nicotinamido)-propanoate was synthesized by reacting O-tert-butyl-L-serine tert-butyl ester hydrochloride and nicotinic acid in the presence of triethylamine (Et3N) in MTBE and acetonitrile. The reaction mixture was charged with propylphosphonic anhydride (T₃P) in acetonitrile and stirred. The resulting slurry was quenched with water and the organic layer washed with an aqueous sodium bicarbonate solution, twice with an aqueous ammonium chloride solution and once again with water. The final MTBE solution was distilled to reduce water content. The (S)-tert-butyl 3-(tert-butoxy)-2-(nicotinamido)-propanoate -MTBE solution was crystallized using MTBE and n-heptane to yield S)-tert-butyl 3-(tert-butoxy)-2-(nicotinamido)-propanoate as an isolated solid.

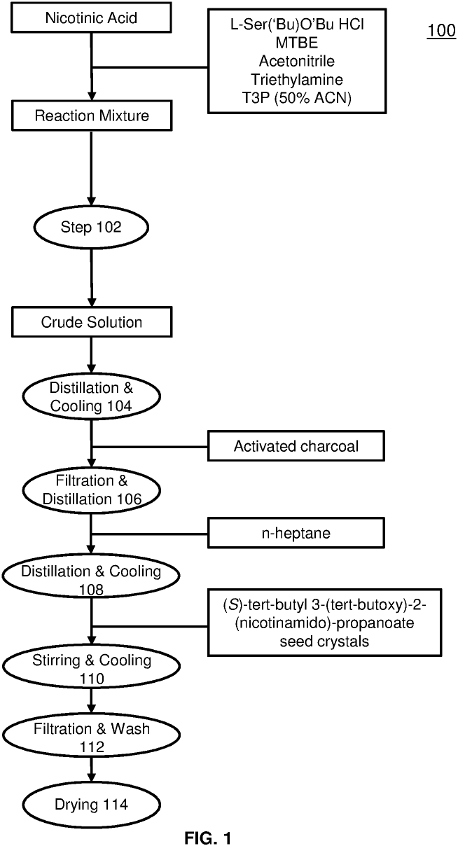

FIG. 1 illustrates a flow diagram of (S)-tert-butyl 3-(tert-butoxy)-2-(nicotinamido)-propanoate synthesis 100, according to some aspects. Nicotinic acid is reacted with L-Ser(‘Bu)O’Bu HCl (O-tert-butyl-L-serine tert-butyl ester hydrochloride) in the presence of triethylamine in MTBE and acetonitrile. The reaction is then charged with T₃P in 50% acetonitrile and stirred to produce a reaction mixture. In step 102, the completed reaction is quenched with water and the aqueous phase extracted with MTBE. The organic layer is washed with Na₂CO₃, twice with NH₄Cl, and once with water to produce a crude solution. The crude solution subsequently undergoes distillation and cooling step 104, filtration and distillation step 106 with activated charcoal, and distillation and cooling step 108 with n-heptane. (S)-tert-butyl 3-(tert-butoxy)-2-(nicotinamido)-propanoate seed crystals are added in stirring and cooling step 110 to initiate crystallization. Following, filtration and wash step 112 and drying step 114, S)-tert-butyl 3-(tert-butoxy)-2-(nicotinamido)-propanoate is produced for downstream use.

### Preparation of 1^{st} Intermediate

In some aspects, the 1^{st} serdexmethylphenidate chloride intermediate is prepared according to Scheme 2.

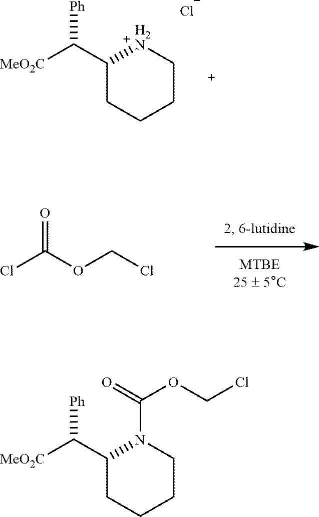

MTBE (349.0 ± 3.0 kg) and 2,6-lutidine (2.8 eq., 52.4 ± 0.5 kg) was added to dexmethylphenidate hydrochloride (d-MPH) (1.0 eq., 47.1 ± 0.2 kg) in a reactor. The reaction mixture was stirred (at 20° C.±5° C. for at least 20 minutes) before adding chloromethyl chloroformate (1.6 eq., 35.8 ± 0.3 kg) to the reactor such that the temperature of the reaction mixture did not exceed 30° C. The reaction mixture was stirred for at least 8 hours at 25° C.±5° C. The reaction mixture was then quenched with approximately 3 volumes of water (relative to d-MPH), such that the temperature of the reaction mixture did not exceed 30° C. The reaction mixture was stirred for at least 6 hours at 20±5° C., and the aqueous layer separated. The MTBE layer was washed with 3 volumes of aqueous sodium bicarbonate solution followed by three volumes of water. The MTBE solution was distilled at atmospheric pressure with an internal temperature of ≤ 59° C. to reach approximately 4.3 volumes with respect to d-MPH and cooled to ≤50° C. The MTBE solution was then cooled to 20±5° C. and the water content determined. Distillation was completed when the MTBE solution reached a water content of ≤ 0.2%. The yield was 90-99%.

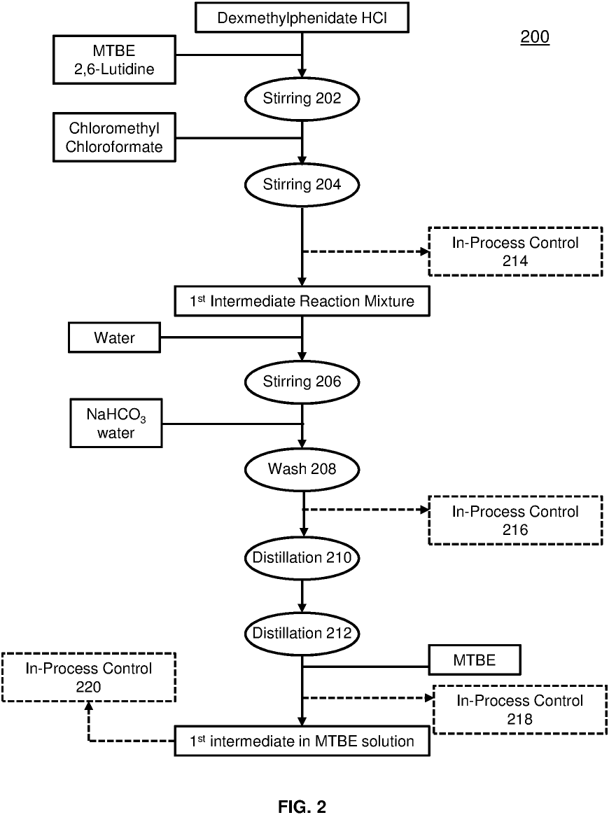

FIG. 2 illustrates a flow diagram of 1^{st} serdexmethylphenidate chloride intermediate (1^{st} SDX intermediate) synthesis 200, according to some aspects. Dexmethylphenidate HCl is charged into a reactor with MTBE and 2,6-lutidine. The resulting reaction mixture can then be stirred in stirring step 202 at 20±5° C. In some aspects, the duration of stirring step 202 can be at least 20 minutes. Chloromethyl chloroformate can is subsequently added to the reactor to generate the 1^{st} intermediate reaction mixture, which can then be stirred in and stirring step 204 at 25±5° C. In some aspects, the duration of stirring step 204 can be at least 8 hours. Upon reaction completion, the 1^{st} intermediate reaction mixture is quenched with water such that the temperature of the 1^{st} intermediate reaction mixture does not exceed 30° C. In stirring step 206, the 1^{st} intermediate reaction mixture can be stirred at 20±5° C. and the aqueous layer separated. In some aspects, the duration of stirring step 206 can be at least 6 hours. In wash step 208, the MTBE layer of the 1^{st} intermediate reaction mixture can be washed with aqueous NaHCO₃ solution and water. In some aspects, the MTBE layer is washed with 3 volumes of NaHCO₃ solution and 3 volumes of water. Completion of wash 208 can be determined by the pH of the final aqueous phase, which is ≥ 6.

In distillation step 210, the MTBE solution/layer of the 1^{st} intermediate reaction mixture is distilled at atmospheric pressure and cooled to ≤50° C. In some aspects, the MTBE solution is distilled to approximately 4 volumes with respect to d-MPH. In distillation step 212, MTBE is added to MTBE solution of the 1^{st} intermediate reaction mixture and distillation repeated. Following distillation 212, the 1^{st} intermediate in MTBE solution is cooled to 20±5° C.

In various aspects, synthesis 200 can have in-process control steps 214, 216, 218, and/or 220. In-process control step 214 can occur between stirring steps 204 and 206, and determines completion of the reaction mixture by HPLC analysis. In some aspects, the reaction is complete when the dexmethylphenidate content is below 4% area with respect to the 1^{st} SDX intermediate. In-process control 216 can occur between wash 208 and distillation 210, and determines the pH of the final aqueous phase. If pH exceeds 6, the MTBE layer is washed again with aqueous NaHCO₃ and water until the pH of the final aqueous phase is ≥ 6. In-process control step 218 occurs after distillation 212, and measures the water content of the 1^{st} intermediate in MTBE solution via Karl Fischer analysis. In some aspects, the resulting water content of the 1^{st} intermediate in MTBE solution is ≤0.2%. If the KF result exceeds 0.2%, the solution can be charged with additional MTBE (150±3.0 kg) and distillation repeated until the water content is ≤ 0.2%. In-process control step 220 analyzes the final 1^{st} intermediate in MTBE solution to determine the wt.-% and mass of the 1^{st} SDX intermediate via HPLC. In some aspects, the yield of 1^{st} SDX intermediate is 90-99%.

### Preparation of 2^{nd} intermediate

In some aspects, the 2^{nd} serdexmethylphenidate chloride intermediate is prepared according to Scheme 3.

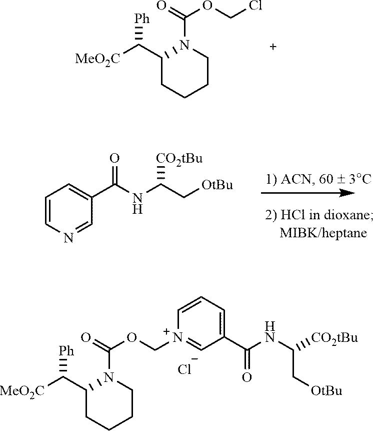

The 1^{st} serdexmethylphenidate chloride intermediate solution (1.2 eq.; 48.0-51.2 kg actual mass of 1^{st} intermediate) was added to (S)-tert-butyl 3-(tert-butoxy)-2-(nicotinamido)-propanoate (1.0 eq., 39.6-42.2 kg) in a reactor and the agitator started. The reaction mixture was charged with 9 volumes of acetonitrile and distilled to approximately 8 volumes under vacuum with an internal temperature of ≤59° C. The solution was then cooled to 20±5° C. and the water content determined. Distillation was completed when the solution reached a water content of ≤ 0.15%. The reaction mixture was heated to 60±3° C. and stirred for at least 45 hours. The reaction was complete when the content of (S)-tert-butyl 3-(tert-butoxy)-2-(nicotinamido)-propanoate was below ≤10% area with respect to the 2^{nd} serdexmethylphenidate chloride intermediate. The reaction mixture was cooled to 20±5° C., charged with 4.0 M HCl solution in dioxane (0.15 eq., 4.85-5.15 kg), and stirred at 20±5° C. for at least 5 minutes. 12 volumes of 4-methyl-2 pentanone (MIBK) were then added to the reaction mixture.

The reaction mixture was distilled at atmospheric pressure with an internal temperature of ≤ 45° C. to remove acetonitrile and MIBK to reach a target of 10 volumes. Following distillation, the reaction mixture temperature was adjusted to 50±5° C. To remove solids, 16 volumes of n-heptane were added over the course of two hours to maintain a reaction temperature of 40-55° C. Once solids were removed, 2^{nd} serdexmethylphenidate chloride intermediate seed crystals ((0.11 wt.% with respect to the theoretical yield of 2^{nd} serdexmethylphenidate chloride intermediate calculated against the quantity of (S)-tert-butyl 3-(tert-butoxy)-2-(nicotinamido)-propanoate charged) were added to the reaction mixture at 50±5° C. to initiate crystallization before charging n-heptane. Following n-heptane addition, the reaction mixture was cooled to 20±5° C., stirred for at least 6 hours, and filtered. The 2^{nd} serdexmethylphenidate chloride intermediate solids were washed with a mixture of MIBK and n-heptane (3:1 volume ratio) and dried (LOD of ≤ 1.0%) at ≤45° C. for at least 12 hours to yield the 2^{nd} serdexmethylphenidate chloride intermediate crystalline solid.

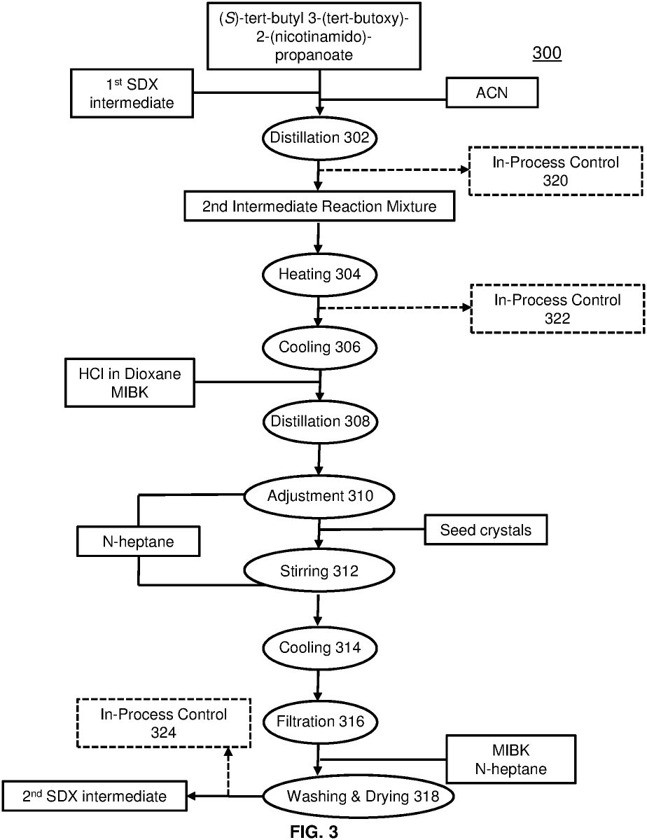

FIG. 3 illustrates a flow diagram of 2^{nd} serdexmethylphenidate chloride intermediate (2^{nd} SDX intermediate) synthesis 300, according to some aspects. (S)-tert-butyl 3-(tert-butoxy)-2-(nicotinamido)-propanoate is charged into a reactor, the 1^{st} serdexmethylphenidate chloride intermediate solution (1^{st} SDX intermediate) is added, and the agitator started. In distillation step 302, the resulting reaction mixture is charged with acetonitrile and distilled under vacuum with an internal temperature of ≤ 59° C. In some aspects, the reaction mixture is charged with 9 volumes of acetonitrile and distilled to approximately 8 volumes. In heating step 304, the reaction is heated to 60±3° C. and stirred for at least 45 hours. In some aspects, the reaction mixture is heated to 59° C. Upon reaction completion, in cooling step 306, the reaction mixture is cooled to 20±5° C., charged with HCl in dioxane, stirred at 20±5° C. for at least 5 minutes, and MIBK added to the reaction mixture. In some aspects, 4.0 M HCl in dioxane can be used and/or 12 volumes of MIBK can be used.

The reaction mixture is then distilled, in distillation step 308, at atmospheric pressure with an internal temperature of ≤ 45° C. to remove acetonitrile and MIBK. In some aspects, the reaction mixture is distilled to reach a target of 10 volumes. Following distillation, the reaction mixture temperature is adjusted to 50±5° C. in adjustment step 310. The reaction mixture is then checked for solids. In some aspects, if solids are detected, n-heptane can be added over at least two hours to maintain a reaction temperature of 40-55° C. If no solids are detected after distillation step 308, 2^{nd} SDX intermediate seed crystals are added to the reaction mixture in stirring step 312 to promote crystallization before charging n-heptane and stirring for at least 5 minutes. The reaction mixture is cooled to 20±5° C. while stirring for at least 6 hours (cooling step 314) and filtered (filtration 316). In washing and drying step 318, the 2^{nd} SDX intermediate solids are washed with MIBK and n-heptane and dried at ≤ 45° C. for at least 12 hours to yield the 2^{nd} SDX intermediate as a crystalline solid. In some aspects, the 2ⁿ^{d} SDX intermediate solids are washed at a 3:1 ratio of MIBK to n-heptane. In some aspects the target drying temperature is 40-45° C.

In various aspects, synthesis 300 can have in-process control steps 320, 322, and/or 324. In-process control step 320 can occur between distillation step 302 and heating step 304, and measures the water content of the reaction mixture via Karl Fischer analysis. In some aspects, distillation is considered complete when the water content of the 2^{nd} intermediate reaction mixture is ≤ 0.15%. If the KF result exceeds 0.15%, the solution can be charged with additional acetonitrile and distillation repeated until the water content is ≤ 0.15%. In some aspects, the solution is charged with 2.5 volumes of acetonitrile. In-process control step 322 determines completion of the reaction mixture by HPLC. In some aspects, the reaction is complete when the content of (S)-tert-butyl 3-(tert-butoxy)-2-(nicotinamido)-propanoate is ≤ 10.0% area with respect to the 2^{nd} SDX intermediate. In some aspects, if the sample does not meet the in-process criteria, stirring can continue at 60±3° C. for at least 4 hours prior to resampling. In-process control step 324 determines loss of drying for the 2^{nd} SDX intermediate solids. In some aspects, the yield of 2^{nd} SDX intermediate as a crystalline solid is 70-85%.

### Preparation of Crude Serdexmethylphenidate Chloride

In some aspects, crude serdexmethylphenidate chloride is prepared according to Scheme 4.

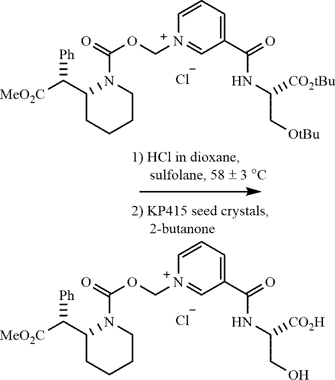

Anhydrous1,4 dioxane (3.4 volumes) and sulfolane (4.6 volumes) was added to 2^{nd} serdexmethylphenidate chloride intermediate crystalline solid (63.5-68.8 kg) in a reactor and the agitator started. 4.0 M HCl in dioxane (2.15 eq., 53.98-59.86 kg) was added and the reaction mixture heated to 58±3° C., stirred between 12 to 18 hours, and cooled to 20-25° C. Upon reaction completion, the reaction mixture was heated to 40-45° C. and 2-butanone was added. The reaction mixture was charged with SDX seed crystals (0.11 wt.% with respect to the theoretical yield of crude SDX when calculated against 2^{nd} serdexmethylphenidate chloride intermediate crystalline solid) and stirred for at least 15 minutes at 40-45° C. To remove solids, additional 2-butanone (19.2 volumes) was added over the course of 3 hours to facilitate precipitation. Once solids were removed, the reaction mixture was cooled to 37-39° C. and SDX seed crystals (0.11 wt.%) were added before charging with additional 2-butanone. The reaction mixture was cooled to ≤ 10° C. over a period of three hours and stirred at ≤ 10° C. for 2-8 hours. The resulting solids were filtered, washed with approximately 2 volumes of 2-butanone, and dried at ≤ 50° C. for at least 10 hours to yield crude SDX as a crystalline solid (LOD of ≤ 1.0%). The yield of isolated crude SDX solids was 60-75%.

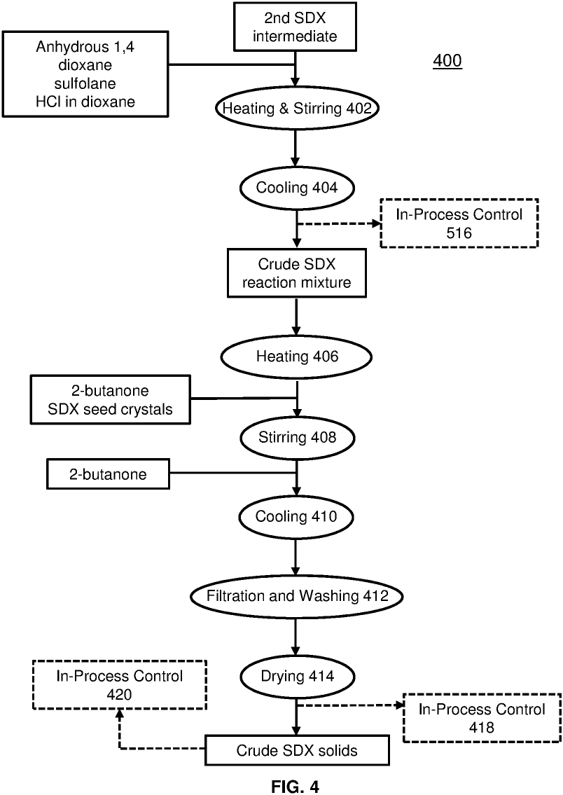

FIG. 4 illustrates a flow diagram of crude serdexmethylphenidate chloride synthesis, according to some aspects. The 2^{nd} serdexmethylphenidate chloride intermediate solution (2^{nd} SDX intermediate) is charged into a reactor and anhydrous 1,4-dioxane and sulfolane are added and the agitator is started. In some aspects, the anhydrous 1,4-dioxane and sulfolane are added in 3.4 volumes and 4.6 volumes, respectively. HCl in dioxane is subsequently added. In some aspects, 4.0 M HCl in dioxane is used. In heating and stirring step 402, the reaction mixture is heated to 58±3° C. and stirred between 12 to 18 hours. In some aspects, the reaction mixture is heated to 59° C. and stirred for 14 hours. The reaction mixture is cooled to 20-25° C. in cooling step 404 to produce the crude SDX reaction mixture. Upon reaction completion, the crude SDX reaction mixture is heated to 40-45° C. in heating step 406 and 2-butanone is added. In some aspects, the crude SDX reaction mixture is heated to 41° C. Immediately following, the reaction mixture is charged with SDX seed crystals.

In stirring step 408, the reaction mixture is stirred for at least 15 minutes at 40-45° C. and checked for the presence of solids. If solids are present, additional 2-butanone is added over a period of at least 3 hours to facilitate precipitation. If no solids are present, the reaction is cooled to 37-39° C. and additional SDX seed crystals are added to the reaction mixture to initiate crystallization before charging with additional 2-butanone. The reaction mixture is subsequently cooled to ≤ 10° C. in cooling step 410 for at least 3 hours and then stirred at ≤ 10° C. for 2-8 hours. The resulting solids are filtered and washed with 2-butanone in filtration step 412. In some aspects, 2 volumes of 2-butanone can be used. In drying step 414, the crude SDX solid is dried. In some aspects, the target drying temperature can be 47° C.

In various aspects, synthesis 400 can have in-process control steps 416, 418, and 420. In-process control step 416 can occur after cooling 404, and determines completion of the reaction by HPLC analysis. In some aspects, the reaction is complete when the combined area of mono-t-butyl ether and mono-t-butyl ester intermediates of the reaction mixture are below ≤ 2.3% with respect to SDX. In some aspects, if the sample does not meet this criteria, the reaction mixture is re-heated, stirred for 2, 4, or 6 hours, and cooled prior to resampling. If the sample still does not meet the criteria, additional HCl in dioxane is added and the reaction mixture heated to 58±3° C. to achieve reaction completion. In-process control step 418 determines loss of drying for the crude SDX solids via USP731. In some aspects, drying is complete when the LOD is ≤ 1.0%. If the LOD exceeds 1.0%, drying can be continued at 47° C. In-process control 420 examines the impurity profile of the resulting crude SDX solids via HPLC prior to purification. In some aspects, the yield of crude SDX solids is 60-75%.

### Purification of Crude Serdexmethylphenidate Chloride

In some aspects, purified, isolated serdexmethylphenidate chloride is prepared according to Scheme 5.

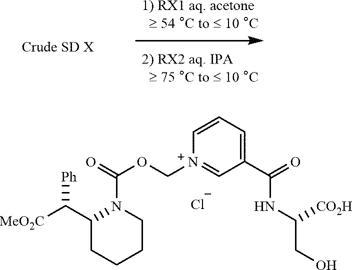

### FIRST RECRYSTALLIZATION (“RX1”

Acetone (6.3 volumes) and water (0.69 volumes) were added to crude SDX solids (37.1-39.8 kg) in a reactor and stirred. The mixture was heated to reflux (≥ 54° C.) and stirred for at least 20 minutes until the solids dissolved. Following dissolution, the solution was adjusted to 45-48° C. and transferred through a filter cartridge. The solution was then cooled to 38-45° C. and SDX seed crystals (0.15 wt.% with respect to crude SDX input) were added to initiate crystallization. The mixture was stirred for at least 15 minutes at 38-45° C. To remove solids, additional acetone (18 volumes) was added over a period of at least 5 hours while cooling to 20±5° C. Once solids were removed, the mixture was cooled to ≤ 10° C. over a period of 2 hours and stirred for at least 2 hours prior to vacuum filtration to isolate SDX RX1 solids. The resulting solids were filtered, washed twice with acetone (3 volumes each), and analyzed for impurities by HPLC. The isolated SDX RX1 solids were dried at ≤ 50° C. for at least 10 hours and analyzed for residual acetone by GC. Drying was complete when residual acetone was ≤ 4500 ppm. The yield of SDX RX1 solids was 75-85%.

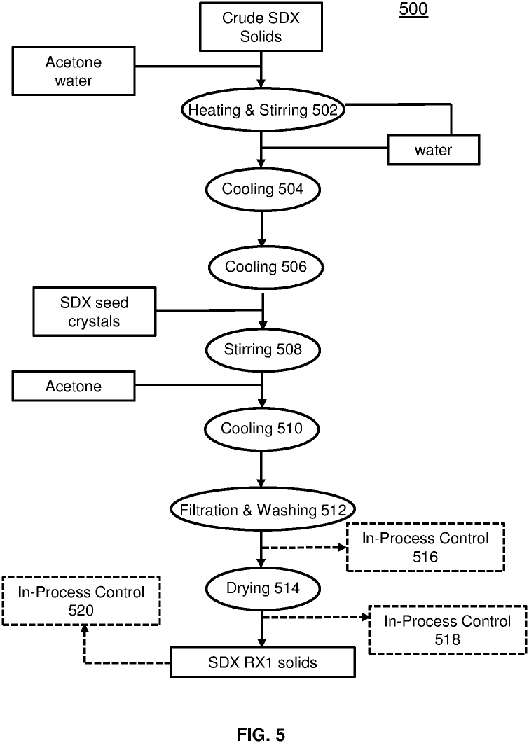

FIG. 5 illustrates a flow diagram of a first recrystallization 500 for purification and isolation of an SDX drug substance, according to some aspects. The crude SDX solids are charged into a reactor, acetone and water are added, and the resulting mixture is stirred. In some aspects, 6.3 volumes and 0.69 volumes of acetone and water are added, respectively. In heating and stirring step 502, the mixture is heated to reflux (≥ 54° C.) and stirred for at least 20 minutes to dissolve solids. In some aspects, if solids remain out of solution, stirring continues for at least another 20 minutes. If solids are still present, additional water is charged and stirring continues for at least 20 minutes at ≥ 54° C. In some aspects, an additional 0.01 volumes of water are added if solids persist. In cooling step 504, the solution is adjusted to 45-48° C. and transferred through a filter cartridge to another reactor.

In cooling step 506, the filter solution is stirred and further cooled to 38-45° C. and SDX seed crystals are added to initiate the crystallization process. In some aspects, the solution can be cooled to 42° C. in cooling step 506. The mixture is then stirred in stirring step 508 for at least 15 minutes at 38-45° C. and checked for the presence of solids. If solids are present, additional acetone is added over a period of at least 5 hours while cooling to 20±5° C. . If no solids are present, the reaction is cooled to 32-37° C. and additional SDX seed crystals are added to the reaction mixture to promote crystallization before charging with additional acetone. In cooling step 510, the mixture is cooled to ≤ 10° C. over a period of 2 hours and then stirred for at least 2 hours prior to vacuum filtration to isolate SDX RX1 solids. In some aspects, in cooling step 510, the target temperature is 5° C. In filtration and washing step 512, the SDX RX1 solids are filtered and washed twice with acetone. In some aspects, 3 volumes of acetone can be used. In drying step 514, the SDX RX1 solids are dried at ≤ 50° C.

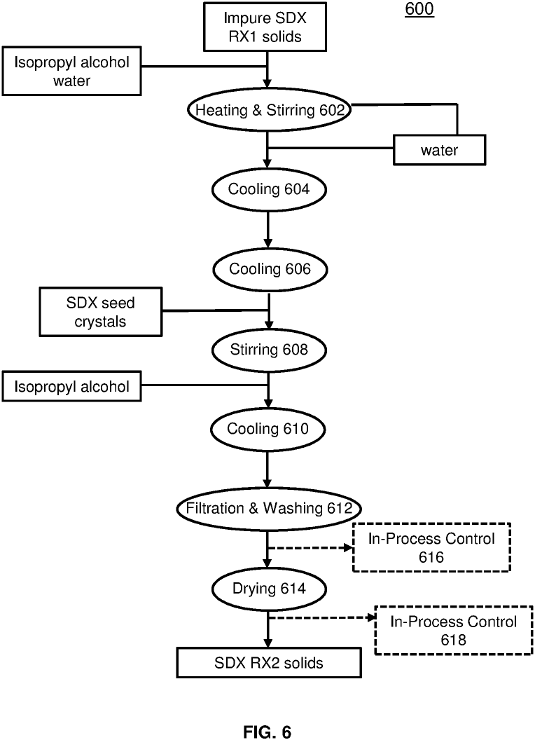

In various aspects, first recrystallization 500 can include in-process control steps 516, 518, and 520. In-process control step 516 can occur after filtration and washing step 512, and determines impurities via HPLC. In some aspects, samples with all specified impurities ≤ 0.15%, all unknown impurities ≤ 0.10%, and total impurities ≤ 1.0%, the isolated SDX RX1 solids are dried at ≤ 50° C. for at least 10 hours and analyzed for residual acetone by GC in in-process control step 518. In some aspects, residual acetone content should be ≤ 4500 ppm. In specified impurities exceed 0.15%, unknown impurities exceed 0.10% and/or total impurities exceed 1.0%, the isolated SDX RX1 solids are dried at ≤ 50° C. for at least 10 hours and subjected to an additional recrystallization procedure (Second Recrystallization; FIG. 6) using isopropyl alcohol. In-process control step 520, determines loss of drying for SDX RX1 solids via USP 731. In some aspects drying is complete when the LOD is ≤ 1.0%. If the LOD exceeds 1.0%, drying can be continued prior to initiation of the second recrystallization step. In some aspects, the yield of pure SDX RX1 solids is 75-85%.

### OPTIONAL SECOND RECRYSTALLIZATION (“RX2”)

Isopropyl alcohol (7.4 volumes) and water (0.60 volumes) were added to impure SDX RX1 solids (28.2-30.4 kg) in a reactor and stirred. The mixture was heated to reflux (≥ 75° C.) and stirred for at least 20 minutes until the solids dissolved. Following dissolution, the solution was adjusted to 63-66° C. and transferred through a filter cartridge. The solution was then cooled to 58-63° C. and SDX seed crystals (0.15 wt.% with respect to SDX RX1 solid input) were added to initiate crystallization.

The mixture was stirred for at least 15 minutes at 58-63° C. To remove solids, additional isopropyl alcohol (13.8 volumes) was added over a period of at least 5 hours while cooling to 25±5° C. If no solids were detected, the mixture was cooled to 52-56° C. before additional SDX seed crystals (0.15 wt%) were added before charging with additional isopropyl alcohol. The mixture was further cooled to ≤ 10° C. over a period of 2 hours and stirred for at least 2 hours prior to vacuum filtration to isolate SDX RX2 solids.

The resulting solids were filtered, washed twice with isopropyl alcohol (3 volumes each), and analyzed for impurities by HPLC. The isolated SDX RX2 solids were dried at ≤ 50° C. for at least 10 hours and analyzed for residual acetone and isopropyl alcohol by GC. Drying was complete when residual acetone and isopropyl alcohol were ≤ 4500 ppm. The yield of SDX RX2 solids, i.e. the purified SDX drug substance, was 84-94%.

FIG. 6 illustrates a flow diagram of a second recrystallization 600 for purification and isolation of an SDX drug substance, according to some aspects. The impure SDX RX1 solids are charged into a reactor, isopropyl alcohol and water are added, and the resulting mixture is stirred. In some aspects, 7.4 volumes and 0.60 volumes of isopropyl alcohol and water are added, respectively. In heating and stirring step 602, the mixture is heated to reflux (≥ 75° C.) and stirred for at least 20 minutes to dissolve solids. In some aspects, if solids remain out of solution, stirring continues for at least another 20 minutes. If solids are still present, additional water is charged and stirring continues for at least 20 minutes at ≥ 75° C. In some aspects, an additional 0.01 volumes of water are added if solids persist. In cooling step 604, the solution is adjusted to 63-66° C. and transferred through a filter cartridge to another reactor.

In cooling step 606, the filter solution is stirred and further cooled to 58-63° C. and SDX seed crystals are added to initiate the crystallization process. In some aspects, the solution can be cooled to 60° C. in cooling step 606. The mixture is then stirred in stirring step 608 for at least 15 minutes at 58-63° C. and checked for the presence of solids. If solids are present, additional isopropyl alcohol is added over a period of at least 5 hours while cooling to 25±5° C. If no solids are present, the reaction is cooled to 52-56° C. and additional SDX seed crystals are added to the reaction mixture to promote crystallization before charging with additional isopropyl alcohol. In some aspects, if no solids are present, the reaction is cooled to 54° C. In cooling step 610, the mixture is cooled to ≤ 10° C. over a period of 2 hours and then stirred for at least 2 hours prior to vacuum filtration to isolate SDX RX2 solids. In some aspects, in cooling step 610, the target temperature is 5° C. In filtration and washing step 612, the SDX RX2 solids are filtered and washed twice with isopropyl alcohol. In some aspects, 3 volumes of isopropyl alcohol can be used. In drying step 614, the SDX RX2 solids are dried at ≤ 50° C.

In various aspects, second recrystallization 600 can include in-process control steps 616 and 618. In-process control step 616 can occur after filtration and washing step 612, and determines impurities via HPLC. In some aspects, when samples with all specified impurities ≤ 0.15%, all unknown impurities ≤ 0.10%, and total impurities ≤ 1.0%, the isolated SDX RX2 solids are dried at ≤ 50° C. for at least 10 hours and analyzed for residual acetone and isopropyl alcohol by GC in in-process control step 618. In some aspects, residual acetone isopropyl content should be ≤ 4500 ppm. In some aspects, the yield of pure SDX RX2 solids is 84-94%.

In some aspects, if residual solvents do not meet the in-process standards (≤ 4500 ppm), the SDX RX2 solids can be subjected to a re-slurry procedure, described below.

### OPTIONAL RE-SLURRY OF CRYSTALLIZED SDX SOLIDS

Isolated SDX RX2 solids and a 3:1 mixture of n-heptane/acetone (12 volumes) were charged into a reactor and the slurry stirred at 20-25° C. for at least 20 hours. The slurry was filtered, washed with a mixture of 5:1 n-heptane/acetone (5 volumes), and dried at ≤ 50° C. for at least 10 hours. An in-sample process was analyzed using GC to confirm residual solvent levels (acetone, n-heptane, and isopropyl alcohol) met in-process standards prior to the batch being discharged from the dryer for final packaging and release testing of SDX drug substance. The yield of purified SDX drug substance was 95-100% after the re-slurry procedure.

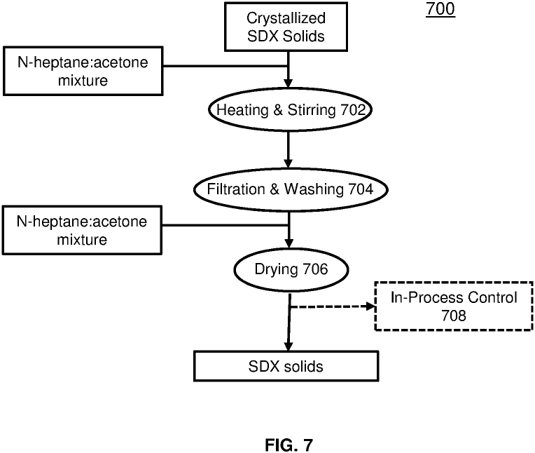

FIG. 7 illustrates re-slurry of crystallized SDX solids 700, according to some aspects. Crystallized SDX solids are charged into a reactor and a mixture of n-heptane/acetone is added. In some aspects, the ratio of n-heptane/acetone is 3:1. In heating and stirring step 702, the slurry is stirred at 20-25° C. for at least 20 hours. The slurry is then filtered in filtration step 704 and washed with a mixture of n-heptane/acetone is added. In some aspects, the ratio of n-heptane/acetone is 5:1. The slurry is then dried at ≤ 50° C. for at least 10 hours to obtain the SDX solids. In some aspects, the slurry is dried at 47° C. In-process control step 708 analyzes residual solvent content via GC. In some aspects, the yield of SDX solids is 95-100%.

In some aspects, reprocessing can occur if the impurity analysis of SDX RX2 solids determines specific impurities exceed 0.15%, unknown impurities exceed 0.10% and/or total impurities exceed 1.0%. In these aspects, the isolated SDX RX2 solids can be subjected to an additional recrystallization using aqueous acetone following the first recrystallization 500 procedure, but using 9 volumes of 91:9 acetone:water and 15 volumes of anti-solvent acetone to further purge residual process impurities.

### Manufacture of Serdexmethylphenidate Chloride and Dexmethylphenidate Hydrochloride Capsules

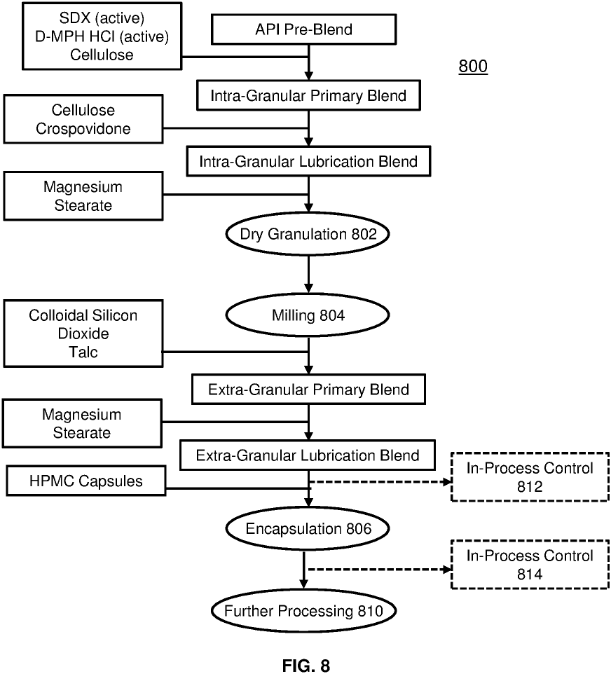

In some aspects, a drug product comprises serdexmethylphenidate chloride and dexmethylphenidate hydrochloride (SDX/d-MPH) in a capsule. In certain aspects, the capsule contains 42 wt% SDX and 9 wt% d-MPH. In some aspects, manufacture method of SDX/d-MPH capsules 800 is shown in FIG. 8:

### PREPARATION OF THE PRE-BLEND

In some aspects, dispensed quantities of SDX and d-MPH drug substances are screened using a vibratory sifter equipped with a 20-mesh screen and added to a tote blender. In some aspects, a portion of microcrystalline cellulose is passed through a 20-mesh screen into the tote blender. In certain aspects, 50% of the batch amount of microcrystalline cellulose is added. The SDX-d-MPH-cellulose pre-blend is mixed. In some aspects, the API pre-blend (blend #1) is mixed for 130 revolutions.

### PREPARATION OF THE INTRA-GRANULAR PRIMARY AND INTRA-GRANULAR LUBRICATION BLEND

In some aspects, the residual portion of microcrystalline cellulose and an amount of crospovidone are passed through a 20-mesh screen and added to the pre-blend and the resulting intra-granular primary blend (blend #2) is mixed. In some aspects, intra-granular primary is missed for 260 revolutions. A portion of magnesium stearate is passed through a 30-mesh screen and added to the blender. In certain aspects, 50% of the batch amount of magnesium stearate is added. The resulting intra-granular lubrication blend (blend #3) is mixed. In some aspects, blend #3 is mixed for 130 revolutions.

### DRY GRANULATION STEP 802 AND MILLING STEP 804

In some aspects, the intra-granular lubrication blend is granulated using roller compaction followed by milling to improve the density and blend flow characteristics of the resulting granules. In certain aspects, a roller compactor configured with two rollers is utilized and the resulting ribbons passed through a screening mill to obtain milled granules for extra-granular blending.

### PREPARATION OF THE EXTRA-GRANULAR PRIMARY AND LUBRICATION BLEND

Following milling 804, a quantity of colloidal silicon dioxide and talc is passed through a 30-mesh screen and added to a blender with the milled granules of the intra-granular lubrication blend. This extra-granular primary blend (blend #4) is mixed and the remaining magnesium stearate is passed through a 30-mesh screen and added to the blender, resulting in extra-granular lubrication blend (blend #5). In some aspects, the extra-granular primary blend is mixed for 260 revolutions. The extra-granular lubrication blend is mixed for another 130 revolutions.

### ENCAPSULATION

The final extra-granular lubrication blend is loaded into an encapsulator product hopper and filled into capsules. In some aspects, the capsules are size 3 HPMC capsules.

In some aspects, further processing steps 810 include capsule dedusting/metal detecting, weight sorting, and/or bulk packaging.

In some aspects, method 800 comprises in-process control step 812, which includes PSD sieve analysis of the extra-granular lubrication blend. In some aspects, in-process control step 814 is part of method 800 and includes appearance inspection and/or weight check of the resulting capsules. The invention is further described in the following paragraphs.

A method for manufacture of a serdexmethylphenidate chloride compound having formula I:

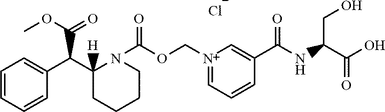 [SMILES: COC(=O)[C@H](c1ccccc1)[C@H]1CCCCN1C(=O)OC[n+]1cccc(C(=O)N[C@@H](CO)C(=O)O)c1 ; [Cl-]]

the method comprising:
(a) synthesizing a compound having formula II:

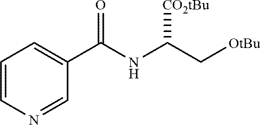 [SMILES: CC(C)(C)OC[C@H](NC(=O)c1cccnc1)C(=O)OC(C)(C)C]

(b) synthesizing a first intermediate compound having formula III:

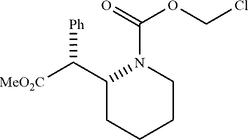 [SMILES: COC(=O)[C@H](c1ccccc1)[C@H]1CCCCN1C(=O)OCCl]

(c) synthesizing a second intermediate compound having formula IV:

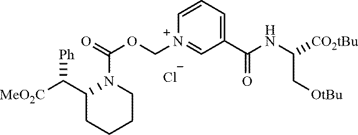 [SMILES: COC(=O)[C@H](c1ccccc1)[C@H]1CCCCN1C(=O)OC[n+]1cccc(C(=O)N[C@@H](COC(C)(C)C)C(=O)OC(C)(C)C)c1 ; [Cl-]]

(d) synthesizing a crude product of the serdexmethylphenidate chloride compound,
(e) purifying the compound having formula V to produce the serdexmethylphenidate chloride compound having formula I.

The method above, wherein synthesis of the compound having formula II comprises reacting O-tert-butyl-L-serine tert-butyl ester hydrochloride and nicotinic acid in the presence of triethylamine in methyl-t-butyl ether and acetonitrile.

The method above , wherein following the reaction of O-tert-butyl-L-serine tert-butyl ester hydrochloride and nicotinic acid in the presence of triethylamine in methyl-t-butyl ether and acetonitrile, the resulting solution is crystallized suing methyl-t-butyl ether and n-heptane to yield the compound having formula II.

The method above, wherein synthesis of the first intermediate compound comprises:
(a) reacting dexmethylphenidate HCl with methyl-t-butyl ether and 2,6-lutidine to obtain a reaction mixture; and
(b) adding chloromethyl chloroformate to the reaction mixture to yield the first intermediate compound.

The method above, wherein synthesis of the second intermediate compound comprises:
(a) reacting the compound having formula II with the first intermediate compound in the presence of acetonitrile, HCl in dioxane, and 4-methyl-2-pentanone to obtain a reaction mixture; and
(b) adding serdexmethylphenidate chloride seed crystals to the reaction mixture to yield the second intermediate compound as a crystalline solid.

The method above, wherein synthesis of the crude product of the serdexmethylphenidate chloride compound having formula V comprises:
(a) reacting the second intermediate crystalline solid with anyhydrous 1,4 dioxane and sulfolane to produce a reaction mixture; and
(b) adding serdexmethylphenidate chloride seed crystals to the reaction mixture to yield the crude product of the serdexmethylphenidate chloride compound having formula V.

The method above, wherein purifying the crude product of the serdexmethylphenidate chloride compound comprises:
(a) reacting the crude product with acetone to produce a reaction mixture; and
(b) adding serdexmethylphenidate chloride seed crystals to the reaction mixture to yield the serdexmethylphenidate chloride compound having formula I as a crystalline solid.

The method above, further comprising:
(f) determining the purity level of the serdexmethylphenidate chloride compound having formula I.

The method above, wherein if impurities are detected, the serdexmethylphenidate chloride compound undergoes an additional purification step comprising:
(a) reacting the serdexmethylphenidate chloride crystalline solid with isopropyl alcohol to produce a reaction mixture; and
(b) adding serdexmethylphenidate chloride seed crystals to the reaction mixture to yield the serdexmethylphenidate chloride compound having formula I as a crystalline solid.

A method of manufacture of a serdexmethylphenidate chloride and dexmethylphenidate hydrochloride capsule comprising:
(a) blending a quantity of serdexmethylphenidate chloride compound having formula I:

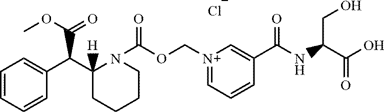 [SMILES: COC(=O)[C@H](c1ccccc1)[C@H]1CCCCN1C(=O)OC[n+]1cccc(C(=O)N[C@@H](CO)C(=O)O)c1 ; [Cl-]]

and a quantity of dexmethylphenidate hydrochloride;
(b) adding a first quantity of microcrystalline cellulose to the blender and mixing to produce a pre-blend;
(c) adding a second quantity of microcrystalline cellulose and a quantify of crospovidone to the pre-blend to produce an intra-granular primary blend;
(d) mixing the intra-granular primary blend;
(e) adding a first quantity of magnesium stearate to the intra-granular primary blend to produce an intra-granular lubrication blend;
(e) mixing the intra-granular lubrication blend;
(f) granulating the intra-granular lubrication blend using a roller compactor;
(g) milling the intra-granular lubrication blend;
(h) adding a quantity of colloidal silicon dioxide and talc to the milled intra-granular lubrication blend to produce an extra-granular primary blend;
(i) mixing the extra-granular primary blend with a second quantity of magnesium stearate to produce an extra-granular lubrication blend;
(j) mixing the extra-granular lubrication blend; and
(k) encapsulating the extra-granular lubrication blend in a capsule.

The method above, wherein the capsule is a size 3 HPMC capsule.

The method above, wherein the pre-blend is mixed for 130 revolutions.

The method above, wherein the intra-granular primary blend is mixed for 260 revolutions.

The method above, wherein the intra-granular lubrication blend is mixed for 130 revolutions.

The method above, wherein the extra-granular primary blend is mixed for 260 revolutions.

The method above, wherein the extra-granular lubrication blend is mixed for 130 revolutions.

The presently described technology is now described in such full, clear, concise and exact terms as to enable any person skilled in the art to which it pertains, to practice the same. It is to be understood that the foregoing describes preferred aspects of the technology and that modifications may be made therein without departing from the spirit or scope of the invention as set forth in the appended claims.

1. A method for manufacture of a serdexmethylphenidate chloride compound having formula I: 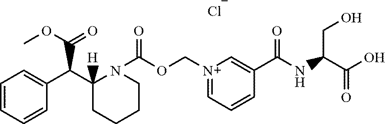 [SMILES: [Cl-] ; COC(=O)[C@H](c1ccccc1)[C@H]1CCCCN1C(=O)OC[n+]1cccc(C(=O)N[C@@H](CO)C(=O)O)c1]
the method comprising:
(a) synthesizing a compound having formula II: 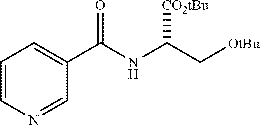 [SMILES: CC(C)(C)OC[C@H](NC(=O)c1cccnc1)C(=O)OC(C)(C)C]
(b) synthesizing a first intermediate compound having formula III: 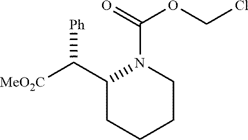 [SMILES: COC(=O)[C@H](c1ccccc1)[C@H]1CCCCN1C(=O)OCCl]
(c) synthesizing a second intermediate compound having formula IV: 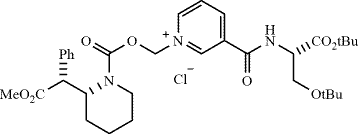 [SMILES: COC(=O)[C@H](c1ccccc1)[C@H]1CCCCN1C(=O)OC[n+]1cccc(C(=O)N[C@@H](COC(C)(C)C)C(=O)OC(C)(C)C)c1 ; [Cl-]]
(d) synthesizing a crude product of the serdexmethylphenidate chloride compound,
(e) purifying the compound having formula V to produce the serdexmethylphenidate chloride compound having formula I.

2. The method of claim 1, further comprising:
(f) determining the purity level of the serdexmethylphenidate chloride compound having formula I.

3. The method of claim 1, wherein if impurities are detected, the serdexmethylphenidate chloride compound undergoes an additional purification step comprising:
(a) reacting the serdexmethylphenidate chloride crystalline solid with isopropyl alcohol to produce a reaction mixture; and
(b) adding serdexmethylphenidate chloride seed crystals to the reaction mixture to yield the serdexmethylphenidate chloride compound having formula I as a crystalline solid.

4. The method of claim 1, wherein synthesis of the compound having formula II comprises reacting O-tert-butyl-L-serine tert-butyl ester hydrochloride and nicotinic acid in the presence of triethylamine in methyl-t-butyl ether and acetonitrile.

5. The method of claim 4, wherein following the reaction of O-tert-butyl-L-serine tert-butyl ester hydrochloride and nicotinic acid in the presence of triethylamine in methyl-t-butyl ether and acetonitrile, the resulting solution is crystallized suing methyl-t-butyl ether and n-heptane to yield the compound having formula II.

6. The method of claim 1, wherein synthesis of the first intermediate compound comprises:
(a) reacting dexmethylphenidate HCl with methyl-t-butyl ether and 2,6-lutidine to obtain a reaction mixture; and
(b) adding chloromethyl chloroformate to the reaction mixture to yield the first intermediate compound.

7. The method of claim 1, wherein synthesis of the second intermediate compound comprises:
(a) reacting the compound having formula II with the first intermediate compound in the presence of acetonitrile, HCl in dioxane, and 4-methyl-2-pentanone to obtain a reaction mixture; and
(b) adding serdexmethylphenidate chloride seed crystals to the reaction mixture to yield the second intermediate compound as a crystalline solid.

8. The method of claim 1, wherein synthesis of the crude product of the serdexmethylphenidate chloride compound having formula V comprises:
(a) reacting the second intermediate crystalline solid with anyhydrous 1,4 dioxane and sulfolane to produce a reaction mixture; and
(b) adding serdexmethylphenidate chloride seed crystals to the reaction mixture to yield the crude product of the serdexmethylphenidate chloride compound having formula V.

9. The method of claim 1, wherein purifying the crude product of the serdexmethylphenidate chloride compound comprises:
(a) reacting the crude product with acetone to produce a reaction mixture; and
(b) adding serdexmethylphenidate chloride seed crystals to the reaction mixture to yield the serdexmethylphenidate chloride compound having formula I as a crystalline solid.

10. A method of manufacture of a serdexmethylphenidate chloride and dexmethylphenidate hydrochloride capsule comprising:
(a) blending a quantity of serdexmethylphenidate chloride compound having formula I: 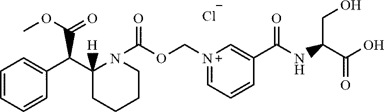 [SMILES: COC(=O)[C@H](c1ccccc1)[C@H]1CCCCN1C(=O)OC[n+]1cccc(C(=O)N[C@@H](CO)C(=O)O)c1 ; [Cl-]]
and a quantity of dexmethylphenidate hydrochloride;
(b) adding a first quantity of microcrystalline cellulose to the blender and mixing to produce a pre-blend;
(c) adding a second quantity of microcrystalline cellulose and a quantify of crospovidone to the pre-blend to produce an intra-granular primary blend;
(d) mixing the intra-granular primary blend;
(e) adding a first quantity of magnesium stearate to the intra-granular primary blend to produce an intra-granular lubrication blend;
(e) mixing the intra-granular lubrication blend;
(f) granulating the intra-granular lubrication blend using a roller compactor;
(g) milling the intra-granular lubrication blend;
(h) adding a quantity of colloidal silicon dioxide and talc to the milled intra-granular lubrication blend to produce an extra-granular primary blend;
(i) mixing the extra-granular primary blend with a second quantity of magnesium stearate to produce an extra-granular lubrication blend;
(j) mixing the extra-granular lubrication blend; and
(k) encapsulating the extra-granular lubrication blend in a capsule.

11. The method of claim 10, wherein the capsule is a size 3 HPMC capsule.

12. The method of claim 10, wherein the pre-blend is mixed for 130 revolutions.

13. The method of claim 10, wherein the intra-granular primary blend is mixed for 260 revolutions.

14. The method of claim 10, wherein the intra-granular lubrication blend is mixed for 130 revolutions.

15. The method of claim 10, wherein the extra-granular primary blend is mixed for 260 revolutions.

16. The method of claim 10, wherein the extra-granular lubrication blend is mixed for 130 revolutions.
